(19) United States
(12) Patent Application Publication
(10) Pub. No.: US 2017/0081700 A1
(43) Pub. Date: Mar. 23, 2017

(54) METHODS FOR SCREENING FOR INHIBITORS OF COMPLEMENT SERINE PROTEASES

(71) Applicant: ANNEXON, INC., South San Francisco, CA (US)

(72) Inventors: Arnon Rosenthal, Woodside, CA (US); Michael Leviten, Palo Alto, CA (US)

(21) Appl. No.: 15/125,367

(22) PCT Filed: Mar. 19, 2015
(86) PCT No.: PCT/US15/21604

§ 371 Date: Sep. 12, 2016

Related U.S. Application Data
(60) Provisional application No. 61/955,742, filed on Mar. 19, 2014.

(51) Int. Cl. C12Q 1/37; A61K 31/277; A61K 31/196; A61K 31/4155; A61K 31/513

(52) U.S. Cl. CPC C12Q 1/37; A61K 31/4155; A61K 31/513; A61K 31/196; A61K 31/277; G01N 2333/96433

(57) ABSTRACT

The present disclosure relates to methods for screening for inhibitors of complement serine proteases by measuring the interaction of a serine protease with a molecular probe in the presence and absence of test compounds.

### CROSS REFERENCES TO RELATED APPLICATIONS

This application claims the benefit of U.S. Provisional Application No. 61/955,742, filed Mar. 19, 2014, which is hereby incorporated by reference in its entirety.

### BACKGROUND

1. Field

The present disclosure relates generally to methods for determining enzyme activities, and more specifically to methods for high-throughput screening of complement serine proteases.

2. Description of Related Art

The complement system in blood plasma is a major mediator of the innate immune defense and a key player in the body's defense against invading microorganisms. However, the complement system is also involved in the clearance of self-antigens and apoptotic cells, it forms a bridge to adaptive immunity and it plays an important role in inflammation, tissue regeneration, and tumor growth. However, inappropriate or excessive activation of the complement cascade has been linked to many autoimmune, neurodegenerative, and inflammatory diseases, including rheumatoid arthritis, as well as ischaemia/reperfusion injury and cancer. Thus, inhibition of the complement system is viewed as a promising therapeutic approach especially for the treatment of inflammatory diseases resulting from excessive complement activation. In other cases complement activities may be suboptimal or deficient, e.g., as a consequence of a genetic mutation or, secondarily, as the result of another disease phenotype. In these cases it may be desirable to activate the complement cascade to afford sufficient protection against microbial infections.

There are three possible pathways of complement cascade activation: the classical, the alternative, and the lectin pathways. All three pathways are ultimately triggered as a result of the detection of surface structures by pattern-recognition proteins. Activation of the classical and lectin pathways is initiated by supramolecular activation complexes in which these pattern-recognition proteins are associated with serine protease zymogens. In the classical pathway, for example, the recognition subunit C1q associates with the serine protease zymogens C1r and C1s. Similarly, in the lectin pathway, the recognition subunit mannose-binding lectin (MBL) associates with the serine protease zymogens MASP-1, MASP-2, and MASP-3. Activation complex zymogens are activated when complement recognition subunits, such as C1q, bind to their respective activator structures, such as immunoglobulins, on target pathogens or cell debris. Zymogen activation then triggers the downstream complement cascade, including the C3-convertase complexes C3bBb and C4b2a. Accordingly, inhibition of the activator serine proteases such as C1r, C1s, MASP-1, MASP-2, and MASP-3, or of downstream serine proteases, such as Factor 2a, Factor Bb, or Factor D is expected to inhibit downstream complement activation. The protease complexes C3bBb and C4b2a, which contain activated factor B and C2 serine proteases respectively, are viewed as especially attractive drug targets, because they generate the inflammatory peptides C3a and C5a and therefore play an important role in amplifying inflammatory processes.

Activation of certain complement serine proteases results in the suppression rather than the activation of the complement cascade. For example, activation of the serine protease Factor I (FI, serum concentration 35 mg/L) is known to inhibit all three complement pathways (see, e.g., Catterall C. F., Lyons A., Sim R. B., Day A. J., Harris T. J., Characterization of primary amino acid sequence of human complement control protein factor I from an analysis of cDNA clones. Biochem. J. 242, 849-856 (1987); Malm S., Jusko M., Eick S., Potempa J., Riesbeck K., et al., Acquisition of Complement Inhibitor Serine Protease Factor I and Its Cofactors C4b-Binding Protein and Factor H by Prevotella intermedia. PLoS ONE 7, e34852. (2012)). FI activity requires the presence of cofactors such as C4BP and FH. C4BP is found in human plasma at concentrations of ˜200 mg/L while the concentration of FH in human plasma varies from 116 to 711 mg/L. C4b-binding protein (C4BP) and factor H (FH) inhibit the classical/lectin or the alternative pathway, respectively, by serving as cofactors in the degradation of C4b and C3b by FI.

FI inhibition is commonly viewed as an attractive drug development strategy for complement deficiency syndromes and, more generally, for treatments aiming at complement activation. For example, the treatment of certain bacterial infections, such as infections with encapsulated bacteria, including Neisseria meningitides, Streptococcus pneumoniae, Haemophilus influenzae, and Neisseria gonorrhoeae, is commonly viewed as benefiting from complement activation. (see, e.g., Figueroa J. E. & Densen P., Infectious diseases associated with complement deficiencies, Clin. Microbiol. Rev. 4, 359-395 (1991)). Certain deficiencies in Factor D, Properdin, C5, C6, C7, C8, or C9 are known to result in predispositions to Neisseria infections. Certain other deficiencies in C1q/r/s, mannose-binding lectin (MBL), C2, C4, C3, or FI are known to result in susceptibility to Gram-positive bacterial infections. Certain mutations in C1q/r/s, C2, C4, C3 and FI are known to cause glomerulonephritis (see, e.g., Blom A. M., Complement: Deficiency Diseases. (2010)). Other diseases commonly viewed as benefiting from complement activation include autoimmune disorders such as systemic lupus erythematosus and immune complexes disorders such as, glomerulonephritis including membranoproliferative glomerulonephritis type II.

Although many complement serine proteases are targets of concerted drug discovery efforts, it has been notoriously difficult in the past to identify promising lead molecules that inhibit their protease targets with sufficient potency and selectivity and that have the pharmacokinetic properties required to serve as viable leads for further preclinical and clinical development. Existing protease inhibitors were typically identified by structure-based or other rational drug design approaches and are commonly based on peptidomimetic scaffolds. See, e.g., Gál et al., Adv. Exp. Med. Biol., 2013, 734, 23-40; Buerke et al., J. Immunol., 2001, 167, 5375-5380; Qu H., Ricklin, D., & Lambertis J. D., Mol. Immunol., 2009, 47, 185-195.

High-throughput screening (HTS) of large and structurally diverse chemical compound libraries has proven invaluable in the identification of novel and structurally diverse starting points for subsequent medicinal chemistry and drug development campaigns. However, it has been challenging in many cases to develop robust assays that can be screened rapidly and cost effectively against compound collections frequently containing on the order of several million molecules. For example, with respect to complement serine proteases, several low-throughput enzymatic assay formats are available to measure protease activities, but robust HTS assays are currently unavailable for many protease targets. This lack of robust and cost-effective high-throughput screening (HTS) assays is generally viewed as a severe bottleneck in the protease targeted drug discovery process.

Thus, there exists a need to devise robust and cost-effective HTS assays for complement serine proteases such as C1s, C1r, MASP-1, MASP-2, MASP-3, Factor 2a, Factor Bb, or Factor D. Such HTS assays could be used to screen large compound collections to identify complement serine protease inhibitors that may serve as starting points for the development of future anti-inflammatory drugs.

All references cited herein, including patent applications and publications, are incorporated by reference in their entirety.

### BRIEF SUMMARY

The present disclosure provides methods for screening inhibitors of a complement serine protease by measuring the level of interaction of the protease with a molecular probe in the presence and absence of a test compound. The disclosure further provides methods for treating disease conditions resulting from the excessive activation of the complement system or for treating disease conditions by activating the complement system by administering therapeutically effective doses of a complement serine inhibitor identified in a screen according to this disclosure.

Accordingly, the present disclosure relates to a method of screening for inhibitors of a complement serine protease, by a) contacting the protease with a molecular probe in the presence and absence of a test compound; and b) measuring the level of interaction of the protease with the molecular probe, whereby a reduction in the interaction in the presence of the test compound compared to the absence of the test compound indicates that the test compound is an inhibitor of the protease. The present disclosure further relates to a method of screening for inhibitors of a complement serine protease, by: a) contacting a complement serine protease with a molecular probe in the presence and absence of a test compound; and b) measuring the level of interaction of the serine protease with the molecular probe, wherein a reduction in the interaction in the presence of the test compound compared to the absence of the test compound indicates that the test compound is an inhibitor of the serine protease.

In some embodiments, the protease is contacted with at least 100, 500, 1,000, 5,000, 10,000, 50,000, 100,000, 500,000, 1,000,000, 2,000,000, 2,500,000, or 3,000,000 test compounds within a 24 hour time period. In some embodiments, the Z-factor of the assay is greater than 0.5, 0.6, 0.7, 0.8, or 0.9.

In some embodiments, inhibition of the protease inhibits the complement cascade. In other embodiments, inhibition of the protease activates the complement cascade. In some embodiments, the complement serine protease is selected from the group consisting of C1s, C1r, MASP1, MASP2, MASP3, Factor 2a, Factor Bb, Factor D, or Factor I. In some embodiments, the complement serine protease is a human, rat, rabbit, mouse, monkey, dog, cat, cow, horse, camel, sheep, goat, or pig protease. In some embodiments, the serine protease is a purified protein. In other embodiments, the serine protease is provided in blood plasma. In some embodiments, the serine protease is provided in an inactive form and activated prior to execution of step b). In some embodiments, the serine protease is a recombinant protein.

In some embodiments, the molecular probe is a protease substrate. In some embodiments, binds to the active site of the protease in a non-covalent manner. In other embodiments, the molecular probe binds the active site of the protease and forms a covalent bond with the protease. In some embodiments, the molecular probe comprises a fluorophosphonate (FP) group. In certain embodiments, the molecular probe is TAMRA-FP, desthiobiotin-FP or azido-FP. In some embodiments, the molecular probe comprises a fluorescent dye, an azido-group, a biotin, or a biotin-analog residue.

In some embodiments, the protease is contacted with the molecular probe in a homogeneous phase. In some embodiments, the protease is contacted with the test compound first and the molecular probe second. In some embodiments, the protease is contacted with the test compound and the molecular probe at the same time.

In some embodiments, the test compound is at least two test compounds. In some embodiments, the test compound is a pool of at least 3, 4, 5, 6, 7, 8, 9, or 10 test compounds. In some embodiments, the test compound is a small molecule. In some embodiments, the test compound is a control compound. In certain embodiments, the control compound is C1s-INH-238 or BCX-1470.

In some embodiments, the protease is contacted with a molecular probe immobilized on a surface. In other embodiments, the protease is contacted with the molecular probe in a microtiter plate. In certain embodiments, the microtiter plate is a 96-well, 384-well, 1,536-well, or 3,456-well microtiter plate. In some embodiments, the protease is contacted with the molecular probe in a total volume of less than 100 μl, 50 μl, 25 μl, 20 μl, 15 μl, 10 μl, or 5 μl. In certain embodiments, the protease is contacted with the molecular probe using an automated liquid handling device.

In some embodiments, the interaction of the protease with the molecular probe is measured using fluorescence polarization, fluorescence intensity, fluorescence resonance energy transfer, or time-resolved fluorescence resonance energy based measurements. In some embodiments, the interaction is measured continuously. In other embodiments, the interaction is measured at one or more time-points.

In some embodiments that may be combined with any of the preceding embodiments, the serine protease is contacted with at least 100, 500, 1,000, 5,000, 10,000, 50,000, 100,000, 500,000, 1,000,000, 1,500,000, 2,000,000, 2,500,000, or 3,000,000 test compounds within a 24 hour time period. In some embodiments that may be combined with any of the preceding embodiments, the method comprises an assay Z-factor value that is greater than 0.5, 0.6, 0.7, 0.8, or 0.9. In some embodiments that may be combined with any of the preceding embodiments, inhibition of the serine protease inhibits the complement cascade. In some embodiments that may be combined with any of the preceding embodiments, inhibition of the serine protease activates the complement cascade. In some embodiments that may be combined with any of the preceding embodiments, the serine protease is selected from C1s, C1r, MASP-1, MASP-2, MASP-3, C2, C2a, C4bC2a, C4b2a3b, C3bBbC3b, C3 convertase, C5 convertase, Factor 2a, Factor Bb, Factor D, and Factor I. In some embodiments that may be combined with any of the preceding embodiments, the serine protease is a human, rat, rabbit, mouse, monkey, dog, cat, cow, horse, camel, sheep, goat, or pig protease. In some embodiments that may be combined with any of the preceding embodiments, the serine protease is a purified protein. In some embodiments that may be combined with any of the preceding embodiments, the serine protease is provided in blood plasma. In some embodiments that may be combined with any of the preceding embodiments, the serine protease is provided in an inactive form and activated prior to execution of step b). In some embodiments that may be combined with any of the preceding embodiments, the serine protease is a recombinant protein. In some embodiments that may be combined with any of the preceding embodiments, the molecular probe is a protease substrate. In some embodiments that may be combined with any of the preceding embodiments, the molecular probe binds the active site of the serine protease in a non-covalent manner. In some embodiments that may be combined with any of the preceding embodiments, the molecular probe binds the active site of the serine protease and forms a covalent bond with the serine protease. In some embodiments that may be combined with any of the preceding embodiments, the molecular probe comprises a fluorophore. In some embodiments that may be combined with any of the preceding embodiments, the fluorophore is a fluorescent protein or peptide. In some embodiments that may be combined with any of the preceding embodiments, the fluorescent protein or peptide is selected from GFP, RFP, YFP, CFP, and derivatives thereof. In some embodiments that may be combined with any of the preceding embodiments, the fluorophore is a non-protein organic fluorophore. In some embodiments that may be combined with any of the preceding embodiments, the non-protein organic fluorophore is selected from a xanthene derivative, a squaraine derivative, a naphthalene derivative, a cyanine derivative, a coumarin derivative, a pyrene derivative, an anthracene derivative, an oxadiazole derivative, an acridine derivative, a tetrapyrrole derivative, an arylmethine derivative, and an oxazine derivative. In some embodiments that may be combined with any of the preceding embodiments, the fluorophore is a quantum dot. In some embodiments that may be combined with any of the preceding embodiments, the fluorophore is a fluorophosphonate (FP) group. In some embodiments that may be combined with any of the preceding embodiments, the molecular probe is TAMRA-FP, desthiobiotin-FP or azido-FP. In some embodiments that may be combined with any of the preceding embodiments, the molecular probe comprises a non-fluorescent detection moiety. In some embodiments that may be combined with any of the preceding embodiments, the non-fluorescent detection moiety is a luminescent or bioluminescent moiety. In some embodiments that may be combined with any of the preceding embodiments, the luminescent or bioluminescent moiety is a luciferase, or derivative thereof. In some embodiments that may be combined with any of the preceding embodiments, the molecular probe comprises a fluorescent dye, an azido-group, a biotin, a biotin-analog residue, radionuclide detection label, a chelating ligand that chelates a detectable label, or an enzyme-substrate label. In some embodiments that may be combined with any of the preceding embodiments, the protease is contacted with the molecular probe in a homogeneous phase. In some embodiments that may be combined with any of the preceding embodiments, the protease is contacted with the test compound first and the molecular probe second. In some embodiments that may be combined with any of the preceding embodiments, the protease is contacted with the test compound and the molecular probe at the same time. In some embodiments that may be combined with any of the preceding embodiments, the test compound is at least two test compounds. In some embodiments that may be combined with any of the preceding embodiments, the test compound is a pool of at least 3, 4, 5, 6, 7, 8, 9, or 10 test compounds. In some embodiments that may be combined with any of the preceding embodiments, the test compound is a control compound. In some embodiments that may be combined with any of the preceding embodiments, the control compound is C1s-INH-238 or BCX-1470. In some embodiments that may be combined with any of the preceding embodiments, the serine protease is contacted with a molecular probe immobilized on a surface. In some embodiments that may be combined with any of the preceding embodiments, the serine protease is contacted with the molecular probe in a microtiter plate. In some embodiments that may be combined with any of the preceding embodiments, the microtiter plate is a 96-well, 384-well, 536-well, or 3,456-well microtiter plate. In some embodiments that may be combined with any of the preceding embodiments, the serine protease is contacted with the molecular probe in a total volume of less than 100 μl, 50 μl, 25 μl, 20 μl, 15 μl, 10 μl, or 5 μl. In some embodiments that may be combined with any of the preceding embodiments, the serine protease is contacted with the molecular probe using an automated liquid handling device. In some embodiments that may be combined with any of the preceding embodiments, the interaction of the serine protease with the molecular probe is measured using fluorescence polarization, fluorescence intensity, fluorescence resonance energy transfer, or time-resolved fluorescence resonance energy based measurements. In some embodiments that may be combined with any of the preceding embodiments, the interaction of the serine protease with the molecular probe is measured continuously. In some embodiments that may be combined with any of the preceding embodiments, the interaction of the serine protease with the molecular probe is measured at one or more time-points. In some embodiments that may be combined with any of the preceding embodiments, the test compound is a small molecule.

The present disclosure also relates to a complement serine protease inhibitor identified by the method of any one of the preceding embodiments.

In some embodiments that may be combined with any of the preceding embodiments, the complement serine protease inhibitor comprises a carbamate chemotype, an acyl-pyrazole chemotype, or a thiophenyl functionality. In some embodiments that may be combined with any of the preceding embodiments, the complement serine protease inhibitor is selected from CD00825, GK00797, KM09391, PHG00507, and JFD00044.

Additionally, the present disclosure relates to a method of treating a disease condition resulting from the excessive activation of the complement system in a subject in need of such treatment, the method comprising the step of administering a therapeutically effective dose of a complement serine protease inhibitor identified in a screen according to a method of this disclosure and optionally repeating said step until no further therapeutic benefit is obtained. The present disclosure also relates to a method of treating a disease condition associated with the excessive activation of the complement system in a subject in need of such treatment, by administering a therapeutically effective dose of a complement serine protease inhibitor of any of the preceding embodiments, wherein the complement serine protease inhibitor inhibits activation of the complement system. The present disclosure also relates to a complement serine protease inhibitor of any of the preceding embodiments for use in treating a disease condition associated with the excessive activation of the complement system in a subject in need of such treatment, wherein the complement serine protease inhibitor inhibits activation of the complement system. The present disclosure also relates to use of a complement serine protease inhibitor of any of the preceding embodiments in the manufacture of a medicament for treating a disease condition associated with the excessive activation of the complement system in a subject in need of such treatment, wherein the complement serine protease inhibitor inhibits activation of the complement system.

In some embodiments, the disease condition is an inflammatory disease condition, a neurodegenerative disease condition, or cancer. In other embodiments, the disease condition is rheumatoid arthritis, ischaemia/reperfusion injury, the Arthus reaction, or the reverse passive Arthus reaction. In some embodiments, the inhibitor comprises a carbamate chemotype, an acyl-pyrazole chemotype, or a thiophenyl functionality. In certain embodiments, the inhibitor is CD00825, GK 00797, KM09391, PHG00507, or JFD00044.

In some embodiments that may be combined with any of the preceding embodiments, the disease condition is an inflammatory disease condition, a neurodegenerative disease condition, or cancer. In some embodiments that may be combined with any of the preceding embodiments, the disease condition is rheumatoid arthritis, ischaemia/reperfusion injury, the Arthus reaction, or the reverse passive Arthus reaction. In some embodiments that may be combined with any of the preceding embodiments, the complement serine protease inhibitor comprises a carbamate chemotype, an acyl-pyrazole chemotype, or a thiophenyl functionality. In some embodiments that may be combined with any of the preceding embodiments, the complement serine protease inhibitor is CD00825, GK00797, KM09391, PHG00507, or JFD00044.

Additionally, the present disclosure relates to a method of treating a disease condition by activating the complement system in a subject in need of such treatment, the method comprising the step of administering a therapeutically effective dose of a complement serine protease inhibitor identified in a screen according to a method of this disclosure, and optionally repeating said step until no further therapeutic benefit is obtained. The present disclosure also relates to a method of treating a disease condition associated with complement deficiency in a subject in need of such treatment, the method comprising the step of administering a therapeutically effective dose of a complement serine protease inhibitor of any of the preceding embodiments, wherein the complement serine protease inhibitor in an inhibitor of Factor I. The present disclosure also relates to a complement serine protease inhibitor of any of the preceding embodiments for use in treating a disease condition associated with complement deficiency in a subject in need of such treatment, wherein the complement serine protease inhibitor in an inhibitor of Factor I. The present disclosure also relates to use of a complement serine protease inhibitor of any of the preceding embodiments in the manufacture of a medicament for treating a disease condition associated with complement deficiency in a subject in need of such treatment, wherein the complement serine protease inhibitor in an inhibitor of Factor I.

In some embodiments, the serine protease inhibitor is an inhibitor of Factor I. In some embodiments, the disease condition is a bacterial infection. In certain embodiments, the bacteria are encapsulated bacteria. In certain embodiments, the bacteria are selected from the group consisting of Neisseria meningitides, Streptococcus pneumoniae, Haemophilus influenzae, and Neisseria gonorrhoeae. In some embodiments, the disease condition is systemic lupus erythematosus or an immune complexes disorder. In certain embodiments, the immune complex disorder is membranoproliferative glomerulonephritis type II.

In some embodiments that may be combined with any of the preceding embodiments, the disease condition is a bacterial infection. In some embodiments that may be combined with any of the preceding embodiments, the bacterial infection is a bacterial infection of encapsulated bacteria. In some embodiments that may be combined with any of the preceding embodiments, the encapsulated bacteria are selected from Neisseria meningitides, Streptococcus pneumoniae, Haemophilus influenzae, and Neisseria gonorrhoeae. In some embodiments that may be combined with any of the preceding embodiments, the disease condition is systemic lupus erythematosus or an immune complex disorder. In some embodiments that may be combined with any of the preceding embodiments, the immune complex disorder is membranoproliferative glomerulonephritis type II.

### DESCRIPTION OF THE FIGURES

FIG. 1 depicts a low throughput gel-based fluorophosphonate-tetramethylrhodamine (TAMRA-FP) labeling experiment. The experiment confirms the enzyme activity dependent labeling of C1S and C1R with TAMRA-FP.

FIG. 2 illustrates that TAMRA-FP labeling of C1S can be followed in a homogeneous 384-well plate format by measuring fluorescence polarization.

FIG. 3 depicts an exemplary microtiter plate layout for a C1S fluopol-ABPP HTS assay. According to this layout, the negative control wells in rows 1 and 2 contain active protease enzyme and DMSO; the positive control wells in rows 23 and 24 omit the protease enzyme; the test compound wells in rows 3 through 22 contain the active protease enzyme in the presence of test compounds of unknown activity.

FIGS. 4A and 4B depict results obtained in 384-well plate C1S fluopol-ABPP assays. FIG. 4A shows a C1S activity time-course. No compounds were added in this experiment. A Z-factor of 0.71 was determined at the 20 min time point. FIG. 4B shows the results of a 384-well pilot screen. In this pilot, the C1S fluopol-ABPP assay was used to screen the NIH Validation Set.

FIG. 5A depicts results obtained in a 384-well plate C1S fluopol-ABPP screen of the Maybridge P31-39 compound collection. Five hits were identified at a cutoff of 30% enzyme inhibition, yielding a hit rate of 0.17%. FIG. 5B shows the chemical structures and designations of the five identified hits.

FIG. 6 illustrates a hit validation experiment performed with two of the hits identified in the screen of FIG. 5. The Maybridge compounds GK00797 and KM09391 were shown to inhibit C1S activity in the gel-based assay similar to the assay depicted in FIG. 1. Compounds showing activity in the gel-based C1S assay are considered confirmed or validated C1S inhibitors.

### DETAILED DESCRIPTION

### General Techniques

The techniques and procedures described or referenced herein are generally well understood and commonly employed using conventional methodology by those skilled in the art, such as, for example, the widely utilized methodologies described in Sambrook et al., Molecular Cloning: A Laboratory Manual 3d edition (2001) Cold Spring Harbor Laboratory Press, Cold Spring Harbor, N.Y.; Current Protocols in Molecular Biology (F. M. Ausubel, et al. eds., (2003)); the series Methods in Enzymology (Academic Press, Inc.): PCR 2: A Practical Approach (M. J. MacPherson, B. D. Hames and G. R. Taylor eds. (1995)), Harlow and Lane, eds. (1988) Antibodies, A Laboratory Manual, and Animal Cell Culture (R. I. Freshney, ed. (1987)); Oligonucleotide Synthesis (M. J. Gait, ed., 1984); Methods in Molecular Biology, Humana Press; Cell Biology: A Laboratory Notebook (J. E. Cellis, ed., 1998) Academic Press; Animal Cell Culture (R. I. Freshney), ed., 1987); Introduction to Cell and Tissue Culture (J. P. Mather and P. E. Roberts, 1998) Plenum Press; Cell and Tissue Culture: Laboratory Procedures (A. Doyle, J. B. Griffiths, and D. G. Newell, eds., 1993-8) J. Wiley and Sons; Handbook of Experimental Immunology (D. M. Weir and C. C. Blackwell, eds.); Gene Transfer Vectors for Mammalian Cells (J. M. Miller and M. P. Calos, eds., 1987); PCR: The Polymerase Chain Reaction, (Mullis et al., eds., 1994); Current Protocols in Immunology (J. E. Coligan et al., eds., 1991); Short Protocols in Molecular Biology (Wiley and Sons, 1999); Immunobiology (C. A. Janeway and P. Travers, 1997); Antibodies (P. Finch, 1997); Antibodies: A Practical Approach (D. Catty., ed., IRL Press, 1988-1989); Monoclonal Antibodies: A Practical Approach (P. Shepherd and C. Dean, eds., Oxford University Press, 2000); Using Antibodies: A Laboratory Manual (E. Harlow and D. Lane (Cold Spring Harbor Laboratory Press, 1999); The Antibodies (M. Zanetti and J. D. Capra, eds., Harwood Academic Publishers, 1995); and Cancer: Principles and Practice of Oncology (V. T. DeVita et al., eds., J. B. Lippincott Company, 1993).

### Overview

The present disclosure relates to methods of screening for inhibitors of complement serine proteases, to inhibitors of complement serine proteases identified by such methods, and furthermore to methods for using such newly identified inhibitors for the treatment of diseases associated and/or caused by the excessive activation of the complement system or associated and/or caused by complement deficiency.

Accordingly, the present disclosure provides methods for screening for inhibitors of a complement serine protease by a) contacting a complement serine protease with a molecular probe in the presence and absence of a test compound; and b) measuring the level of interaction of the serine protease with the molecular probe, wherein a reduction in the interaction in the presence of the test compound compared to the absence of the test compound indicates that the test compound is an inhibitor of the serine protease.

The present disclosure also provides complement serine protease inhibitor identified by the screening methods of the present disclosure.

The present disclosure further provides methods of treating a disease condition associated with and/or resulting from the excessive activation of the complement system in a subject in need of such treatment, whereby the method includes the step of administering a therapeutically effective dose of a complement serine protease inhibitor identified in a screen according to this disclosure, and optionally repeating said step until no further therapeutic benefit is obtained.

The present disclosure further provides methods of treating a disease condition associated with and/or resulting from complement deficiency in a subject in need of such treatment, whereby the method includes the step of administering a therapeutically effective dose of a complement serine protease inhibitor identified in a screen according to this disclosure, and optionally repeating said step until no further therapeutic benefit is obtained.

The following description sets forth exemplary methods, parameters and the like. It should be recognized, however, that such description is not intended as a limitation on the scope of the present disclosure but is instead provided as a description of exemplary embodiments.

### Targeted Complement Serine Proteases

The methods of this disclosure can be used to screen for inhibitors of any complement serine protease. Preferred examples of complement serine proteases include, without limitation, C1r, C1s, MASP-1, MASP-2, MASP-3, C2, C2a, C4bC2a, C4b2a3b, C3bBbC3b, C3 convertase, C5 convertase, Factor Bb, Factor 2a, Factor D and Factor I (FI). The complement serine protease may be derived from any organism having a complement system, including human (e.g., NM_001733, NM_201442, NM_139125, NG_007289, AF_284421, NM_001710, NG_011730), rat, rabbit, mouse, monkey, dog, cat, cow, horse, camel, sheep, goat, or pig.

The complement serine protease may be added to the screening assay in an enzymatically active form. Alternatively, the protease may be enzymatically inactive when the screening assay is initiated. For example, the protease may be applied in its zymogen form at the initiation of the assay. Where the protease is initially applied in an inactive form, it may be subsequently activated during the course of the ongoing assay. This protease activation may occur either prior to test compound addition or after addition of the test compound. Moreover, protease activation may be triggered by the addition of another enzymatically active protease, such as an upstream protease in the complement cascade. Alternatively, activation of the complement serine protease may be achieved by triggering the entire upstream complement cascade, e.g., through the addition of immuno-complexes or bacterial cells or fragments to blood plasma or through the use of an in vitro reconstituted complement cascade.

The complement serine protease may be used in a purified or partially purified form. Alternatively, the protease may be contained in blood plasma or in a plasma fraction. The protease may be purified from blood plasma or it may be produced as a recombinant protein. Methods for expressing and purifying recombinant complement serine proteases are well known in the art. Complement proteins can be expressed in a mammalian cell line (see, e.g., Perlmutter D. H., Colten H. R., Grossberger D., Strominger J., Seidman J. G., Chaplin D. D. J., Expression of complement proteins C2 and factor B in transfected L cells. Clin. Invest. 76, 1449-54 (1985)), in bacterial or yeast cells (see, e.g., Schmidt C. Q., Slingsby F. C., Richards A., Barlow P. N., Production of biologically active complement factor H in therapeutically useful quantities. Protein Expr. Purif. 76, 254-63 (2011)), in insect cells, plants, or whole organisms. Complement serine protease may be a wild-type protein or a mutant protein containing, for example, amino acid mutations, deletions, insertions, truncations, or additions, including mutations in the protease domain. Additions may include, e.g., a tag or fusion protein, such as a His-tag, GST-tag, FLAG-tag, SNAP-tag, or other fusion elements that may facilitate protein purification or protein modification, for example with fluorescent dyes or affinity tags, such as biotin-derived affinity tags. In some embodiments, the complement serine protease may be a part of a multiprotein protein complex such as the C3-convertase complexes C3bBb and C4b2a.

### Molecular Probe

The molecular probes of this disclosure can, for example, bind to the catalytic center of a complement serine protease of the present disclosure and undergo a change in their chemical or physical properties as a result of this binding event. Accordingly, in some embodiments, a measurement of the probe's changing chemical or physical properties therefore allows the quantification of the probe's interaction with the protease.

In some embodiments, the molecular probes are protease substrates that are hydrolyzed upon their binding to the protease's catalytic center. Such protease substrates may include, without limitation, peptide or protein substrates or other fluorogenic or colorigenic protease substrates such as Cbz-Gly-Arg-S-bzl. Alternatively, the molecular probes may bind non-covalently to the protease's catalytic center without any turnover occurring. In other embodiments the molecular probe forms a covalent bond with the catalytic center. The covalent bond may be formed with the catalytic Ser of the serine protease or with another amino acid residue in the catalytic center. The covalently binding probes may include fluorophosphonate (FP) groups. Exemplary probes include tetramethylrhodamine-FP (TAMRA-FP), desthiobiotin-FP, and azido-FP. Probes of this type are commercially available. See, e.g., Life Technologies website; see also, Verhelst S. H. L. & Bogyo M., Chemical Proteomics Applied in Target Identification and Drug Discovery, BioTechniques 38, 175-177 (2005).

In some embodiments, a serine protease substrate of the present disclosure is attached to a fluorophore. Any fluorophore known in the art may be used. In some embodiments, the fluorophore may be a fluorescent protein or peptide, including without limitation GFP, RFP, YFP, CFP, derivatives thereof, and the like. In some embodiments, the fluorophore may be a non-protein organic fluorophore, including without limitation a xanthene derivative (e.g., rhodamine, fluorescein, Texas red, etc.), a squaraine derivative, a naphthalene derivative, a cyanine derivative (e.g., cyanine, indocarbocyanine, oxacarbocyanin, etc.), a coumarin derivative, a pyrene derivative, an anthracene derivative, an oxadiazole derivative, an acridine derivative, a tetrapyrrole derivative, an arylmethine derivative, or an oxazine derivative. In some embodiments, the fluorophore may be a quantum dot. Lists of suitable fluorophores and their properties (e.g., absorption and emission spectra, molar extinction coefficient, photobleaching properties, brightness, photostability, and so forth) are commonly obtained through manufacturers, e.g., The Molecular Probes® Handbook, 11^{th} ed. (Life Technologies, Carlsbad, Calif.). In some embodiments, the serine protease substrate is attached to a non-fluorescent detection moiety, such as a luminescent or bioluminescent moiety (e.g., a luciferase such as Renilla luciferase or a derivative thereof), and a bioluminogenic substrate is further included (e.g., a luciferin such as a coelenterazine or coelenterazine derivative, including without limitation DeepBlueC™). In some embodiments, a fluorophore associated with the serine protease substrate may be treated with light of a wavelength sufficient to cause the fluorophore to emit fluorescence. In some embodiments, subsequent fluorescence emitted by the fluorophore associated with the serine protease substrate is detected. Information on the wavelengths of light sufficient to cause a fluorophore of the present disclosure to emit fluorescence and the wavelengths of light emitted by the fluorophore is widely available in the art and typically supplied by the manufacturer (e.g., Life Technologies, Pierce Biotechnology, Thermo Scientific, abcam, etc.).

In some embodiments, the fluorophore may be attached to the serine protease substrate by direct coupling, or they may be indirectly coupled through an intermediary (e.g., antibody binding, biotin:streptavidin binding, an affinity tag, etc.). For example and without limitation, if the fluorophore is a fluorescent protein, the serine protease substrate may be translated with the coding sequence of the fluorescent protein attached (e.g., by a peptide linker) in-frame with the coding sequence of the serine protease substrate, such that a fusion protein is produced. For example and without limitation, if the fluorophore is a non-protein organic fluorophore, the fluorophore may be chemically attached (e.g., through a covalent bond) to the serine protease substrate. Labeling kits for attaching a fluorophore to a protein of interest (e.g., a serine protease substrate of the present disclosure) are commercially available and typically employ a chemical reaction between a primary amine of the protein and an amine-reactive fluorophore or crosslinker.

Any suitable method for detecting fluorescence emitted at the appropriate wavelength (e.g., a wavelength described supra) may be used. Fluorescence detection techniques may employ a plate reader (e.g., a PHERAstar plate reader from BMG LABTECH, Ortenberg, Germany), fluorescence microscope, flow cytometer, or any other equipment known in the art for fluorescence detection.

In some embodiments, molecular probes of the present disclosure may include a radionuclide detection label. Examples of suitable radionuclides (i.e., radioisotopes), include, without limitation, ³H, ¹¹C, ¹⁴C, ¹⁸F, ³²P, ³⁵S, ⁶⁴Cu, ⁶⁸Ga, ⁸⁶Y, ⁹⁹Tc, ¹¹¹In, ¹²³I, ¹²⁴I, ¹²⁵I, ¹³¹I, ¹³³Xe, ¹⁷⁷Lu, ²¹¹At, and ²¹³Bi. The molecular probe can be labeled with ligand reagents that bind, chelate or otherwise complex a radioisotope metal where the reagent is reactive with a suitably reactive group of the molecular probe, using techniques described, for example, in Current Protocols in Immunology, Volumes 1 and 2, Coligen et al, Ed. Wiley-Interscience, New York, N.Y., Pubs. (1991).

Chelating ligands which may complex a metal ion include DOTA, DOTP, DOTMA, DTPA and TETA (Macrocyclics, Dallas, Tex.). Linker reagents such as DOTA-maleimide (4-maleimidobutyramidobenzyl-DOTA) can be prepared by the reaction of aminobenzyl-DOTA with 4-maleimidobutyric acid (Fluka) activated with isopropylchloroformate (Aldrich), following the procedure of Axworthy et al (2000) Proc. Natl. Acad. Sci. USA 97(4): 1802-1807). DOTA-maleimide reagents react with a reactive group of the molecular probe and provide a metal complexing ligand on the antibody (Lewis et al (1998) Bioconj. Chem. 9:72-86). Chelating linker labelling reagents such as DOTA-NHS (1,4,7,10-tetraazacyclododecane-1,4,7,10-tetraacetic acid mono (N-hydroxysuccinimide ester) are commercially available (Macrocyclics, Dallas, Tex.).

Metal-chelate complexes suitable as molecular probe labels, for example, for imaging experiments are disclosed: U.S. Pat. No. 5,342,606; U.S. Pat. No. 5,428,155; U.S. Pat. No. 5,316,757; U.S. Pat. No. 5,480,990; U.S. Pat. No. 5,462,725; U.S. Pat. No. 5,428,139; U.S. Pat. No. 5,385,893; U.S. Pat. No. 5,739,294; U.S. Pat. No. 5,750,660; U.S. Pat. No. 5,834,456; Hnatowich et al (1983) J. Immunol. Methods 65: 147-157; Meares et al (1984) Anal. Biochem. 142:68-78; Mirzadeh et al (1990) Bioconjugate Chem. 1:59-65; Meares et al (1990) J. Cancer 1990, Suppl. 10:21-26; Izard et al (1992) Bioconjugate Chem. 3:346-350; Nikula et al (1995) Nucl. Med. Biol. 22:387-90; Camera et al (1993) Nucl. Med. Biol. 20:955-62; Kukis et al (1998) J. Nucl. Med. 39:2105-2110; Verel et al (2003) J. Nucl. Med. 44: 1663-1670; Camera et al (1994) J. Nucl. Med. 21:640-646; Ruegg et al (1990) Cancer Res. 50:4221-4226; Verel et al (2003) J. Nucl. Med. 44: 1663-1670; Lee et al (2001) Cancer Res. 61:4474-4482; Mitchell, et al (2003) J. Nucl. Med. 44: 1105-1112; Kobayashi et al (1999) Bioconjugate Chem. 10: 103-111; Miederer et al (2004) J. Nucl. Med. 45: 129-137; DeNardo et al (1998) Clinical Cancer Research 4:2483-90; Blend et al (2003) Cancer Biotherapy & Radiopharmaceuticals 18:355-363; Nikula et al (1999) J. Nucl. Med. 40:166-76; Kobayashi et al (1998) J. Nucl. Med. 39:829-36; Mardirossian et al (1993) Nucl. Med. Biol. 20:65-74; Roselli et al (1999) Cancer Biotherapy & Radiopharmaceuticals, 14:209-20.

In some embodiments, molecular probes of the present disclosure may include a fluorescent label. Suitable examples of fluorescent labels include, without limitation, rare earth chelates (europium chelates), fluorescein types including FITC, 5-carboxyfluorescein, 6-carboxy fluorescein; rhodamine types including TAMRA; dansyl; Lissamine; cyanines; phycoerythrins; Texas Red; and analogs thereof. The fluorescent labels can be conjugated to molecular probes using the techniques disclosed in Current Protocols in Immunology, supra, for example. Fluorescent dyes and fluorescent label reagents include those which are commercially available from Invitrogen/Molecular Probes (Eugene, Oreg.) and Pierce Biotechnology, Inc. (Rockford, Ill.).

In some embodiments, molecular probes of the present disclosure may include an enzyme-substrate label. Examples of suitable enzyme-substrate labels are well-know (see, e.g., U.S. Pat. No. 4,275,149). The enzyme generally catalyzes a chemical alteration of a chromogenic substrate that can be measured using various techniques. For example, the enzyme may catalyze a color change in a substrate, which can be measured spectrophotometrically. Alternatively, the enzyme may alter the fluorescence or chemiluminescence of the substrate. Techniques for quantifying a change in fluorescence are described above. The chemiluminescent substrate becomes electronically excited by a chemical reaction and may then emit light which can be measured (using a chemiluminometer, for example) or donates energy to a fluorescent acceptor. Examples of enzymatic labels include, without limitation, luciferases (e.g., firefly luciferase and bacterial luciferase; U.S. Pat. No. 4,737,456), luciferin, 2,3-dihydrophthalazinediones, malate dehydrogenase, urease, peroxidase such as horseradish peroxidase (HRP), alkaline phosphatase (AP), β-galactosidase, glucoamylase, lysozyme, saccharide oxidases (e.g., glucose oxidase, galactose oxidase, and glucose-6-phosphate dehydrogenase), heterocyclic oxidases (such as uricase and xanthine oxidase), lactoperoxidase, microperoxidase, and the like. Techniques for conjugating enzymes to antibodies are described in O'Sullivan et al (1981) “Methods for the Preparation of Enzyme-Antibody Conjugates for use in Enzyme Immunoassay”, in Methods in Enzym. (ed J. Langone & H. Van Vunakis), Academic Press, New York, 73:147-166.

Suitable examples of enzyme-substrate combinations include, without limitation: horseradish peroxidase (HRP) with hydrogen peroxidase as a substrate, wherein the hydrogen peroxidase oxidizes a dye precursor (e.g., orthophenylene diamine (OPD) or 3,3′,5,5′-tetramethylbenzidine hydrochloride (TMB)); alkaline phosphatase (AP) with para-nitrophenyl phosphate as chromogenic substrate; and β-D-galactosidase (β-D-Gal) with a chromogenic substrate (e.g., p-nitrophenyl-β-D-galactosidase) or fluorogenic substrate 4-methylumbelliferyl-β-D-galactosidase. Numerous other enzyme-substrate combinations are available to those skilled in the art. For a general review, see U.S. Pat. No. 4,275,149 and U.S. Pat. No. 4,318,980.

The molecular probes of this disclosure may interact with the serine protease in solution or the probes may be immobilized to a surface. The resulting serine protease assay may therefore be a homogeneous or a heterogeneous assay.

The interaction of the molecular probe with the serine protease may be measured by any suitable method detecting changes in the probe's chemical or physical properties that correlate to protease binding. Such methods include, without limitation, absorbance measurements, fluorescence intensity or fluorescence polarization measurements, fluorescence resonance energy transfer (FRET) or time-resolved fluorescence resonance energy transfer (TR-FRET) measurements, nuclear magnetic resonance (NMR) spectroscopy, mass spectrometry (MS), surface plasmon resonance spectroscopy (SPR), ELISA or others.

In a preferred embodiment, the complement serine protease assay is a fluorescence-polarization-activity-based protein profiling (fluopol-ABPP) assay.

### Test Compound

A test compound is a protease inhibitor if in its presence the interaction between the molecular probe and the complement serine protease is reduced relative to the respective interaction occurring in the absence of the test compound. For example, a test compound is a protease inhibitor if it displaces the molecular probe from the catalytic center of the serine protease or if it can hinder the probe's access to the protease's catalytic center. Protease inhibitors may reduce the interaction between a serine protease and a molecular probe by more than 5%, 10%, 20%, 30%, 40%, 50%, 60%, 70%, 80%, 90%, 95%, or 99% relative to the interaction observed in the absence of the inhibitor.

In some embodiments, the test compounds are small molecules, i.e., their molecular mass is <1,000 Da. Test compounds may include peptides or peptide mimetics. Some test compounds may contain functional groups that can form covalent bonds with the catalytic serine of the serine protease (so-called “serine-traps” or “warheads”). Examples for such test compounds include α-haloketones, α-ketoamides, diketones, heterocyclic ketones, sulfonamido group, boronate esters α-ketoacid, α-amino cyclic boronates, pyrrolidine-5,5-trans-lactam core, and generally compounds described, e.g., in Lin C. 6HCV NS3-4A Serine Protease, Chapter 6 in Hepatitis C Viruses: Genomes and Molecular Biology, Tan S. L., editor. Norfolk (UK), Horizon Bioscience (2006); in Baker S. Z., Ding C. Z., et al., Therapeutic Potential of Boron Containing Compounds, Future Med. Chem. 1, 1275-1288 (2009); and in Li X., Zhang Y. K. et al., Design, Synthesis and SAR of α-Amino Cyclic Boronate-containing Macrocyclic Inhibitors of HCV NS3/4A Serine Protease, Poster No. MEDI 126 of 239th ACS National Meeting, San Francisco, Mar. 21, 2010.

Some test compounds are control compounds that are known inhibitors of a serine protease, such as C1s-INH-248 or BCX-1470 (see, e.g., Buerke M. et al., J. Immunol. 167, 5375-5380 (2001); Szalai A. J. et al., J. Immunol. 164, 463-468 (2000).

Test compounds are generally provided as part of molecular libraries containing more than 100, 500, 1,000, 5,000, 10,000, 50,000, 100,000, 500,000, 1,000,000, 1,500,000, 2,000,000, 2,500,000 or 3,000,000 members. In some embodiments, the molecular libraries are plated in 96-well, 384-well, 1,536-well, or 3,456-well microtiter plates. Some plated libraries may contain only a single test compound per well. Other compound libraries may be arranged in test compound pools, with each pool containing at least 2, 3, 4, 5, 6, 7, 8, 9, or 10 test compounds per well. Typically, test compounds are stored in stock solutions of about 1 mM, 2 mM, 5 mM, or 10 mM concentrations. In some embodiments, test compound stocks are prepared as dilution series. Test compound stock solutions may be prepared in DMSO, DMF, acetone, ethanol, aqueous buffers, or other solutions.

### Assay Protocol and Screening Process

In preferred embodiments of this disclosure, the screen for complement serine protease inhibitors is conducted in a microtiter assay plate. Exemplary assay plates include 96-well, 384-well, 1,536-well, or 3,456-well microtiter plates. In some embodiments, the total assay volume is less than 5 μl, 10 μl, 15 μl, 20 μl, 25 μl, 30 μl, 40 μl, 50 μl, 75 μl, or 100 μl. Test compounds may be screened at single concentrations or in dose-responses. Where compounds are screened at single concentrations, the final concentrations at which test compounds are incubated with the protease may not exceed 100 nM, 1 μM, 2 μM, 5 μM, 10 μM, 20 μM, 50 μM, 75 μM or 100 μM. Where compounds are screened in dose-responses, the highest final concentration at which the test compounds are incubated with the protease may not exceed 100 nM, 1 μM, 2 μM, 5 μM, 10 μM, 20 μM, 50 μM, 75 μM or 100 μM. Preferred dose-response curves include 8-point 3-fold dose response curves (e.g., 10 μM, 3.3 μM, 1 μM, 0.33 μM, 0.1 μM, 0.03 μM, 0.01 μM, 0.003 μM final assay concentrations) and 12 point 3-fold dose-response curves. In some embodiments, the serine protease is screened against a total of more than 100, 500, 1,000, 5,000, 10,000, 50,000, 100,000, 500,000, 1,000,000, 1,500,000, 2,000,000, 2,500,000 or 3,000,000 test compounds. These test compounds may be contacted with the protease individually, e.g., in separate microtiter plate wells, or in pools of up to 2, 3, 4, 5, 7, 8, 9, or 10 compounds. In some embodiments, the screen is completed within a 24 hour time period. In some embodiments, the screen is conducted using automated screening equipment, such as plate handling robotics and automated liquid handling.

The relative order in which the protease, the molecular probe and the test compound are added to the screening assay may vary. In some embodiments, the protease is dispensed first, the test compound second, and the molecular probe third. In other embodiments, a solvent, such as an aqueous reaction buffer, is dispensed first, the test compound is dispensed second, the serine protease third, and the molecular probe fourth. In other embodiments the test compound is dispensed first, the serine protease second and the molecular probe is dispensed last. In some embodiments the test compound is dispensed with the molecular probe at the same time.

The protease may be preincubated with the test compound prior to addition of the molecular probe. In some embodiments, the pre-incubation period is at least 1 minute, 3 minutes, 5 minutes, 10 minutes, 15 minutes, 20 minutes, 30 minutes, 45 minutes, or 1 hour. Additionally, the period of incubation for the protease and the molecular probe may vary and extend to least 1 minute, 3 minutes, 5 minutes, 15 minutes, 20 minutes, 30 minutes, 45 minutes, 1 hour, 1.5 hours, 2 hours, 3 hours, 4 hours, 5 hours, 6 hours, 7 hours, 8 hours, 9 hours, 10 hours, 11 hours, or 12 hours. The level of interaction between the protease and the molecular probe may be monitored continuously, intermittently, e.g., at least every 1 minute, 3 minutes, 5 minutes, 10 minutes, 15 minutes, 20 minutes, 30 minutes, 45 minutes, or 60 minutes, or in the form of an endpoint measurement, i.e., at the beginning and the end of the incubation period.

The robustness of the screening assay is assessed by Z-factor analysis (see, e.g., Zhang et al., J. Biomol. Screen, 1999, 4, 67-73). Briefly, to determine the Z-factor, several experimental iterations of the protease screening assay are conducted in the presence of either a positive control compound or a number of randomized test compounds of unknown activity. Next, the level of interaction between the protease and the molecular probe is measured in the presence of the positive control compound and in the presence of the unknown test compounds respectively. For example, a protease assay is conducted in multiple wells of a microtiter plate, whereby each well contains either a positive control compound or a randomized test compound of unknown activity. Next, the fluorescence polarization signals are collected for each well that quantify the interaction between the molecular probe and the protease in the presence of either the positive control compound, e.g., a known protease inhibitor, or a randomized compound of unknown activity. The Z-factor of the assay is determined according to the following formula:

Z-factor=1−(3SD of TC+3SD of PC)/|mean of TC−mean of PC|

3SD of TC=3-fold standard deviation of signal from test compound experiments

3SD of PC=3-fold standard deviation of signal from positive control experiments

Mean of TC=mean value of signal from test compound experiments

Mean of PC=mean value of signal positive control experiments

According to this formula, Z-factors range between 0 and 1. The robustness of a screening assay increases with increasing Z-factors. A screening assay characterized by a Z-factor of >0.5 is typically considered sufficiently robust to support an HTS screen.

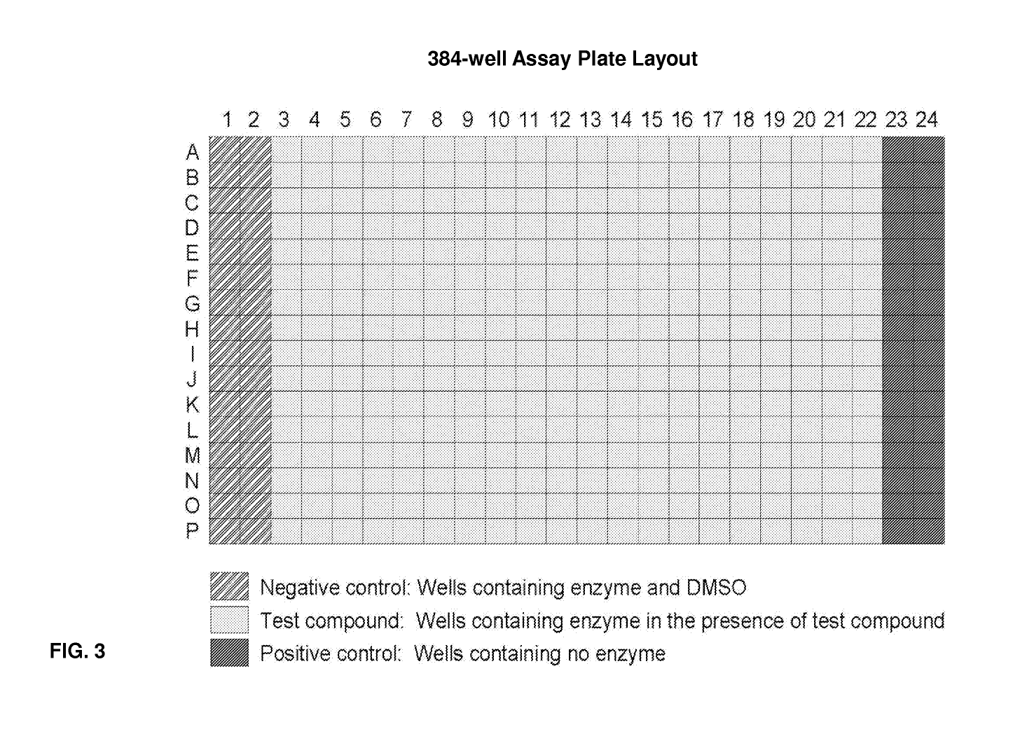

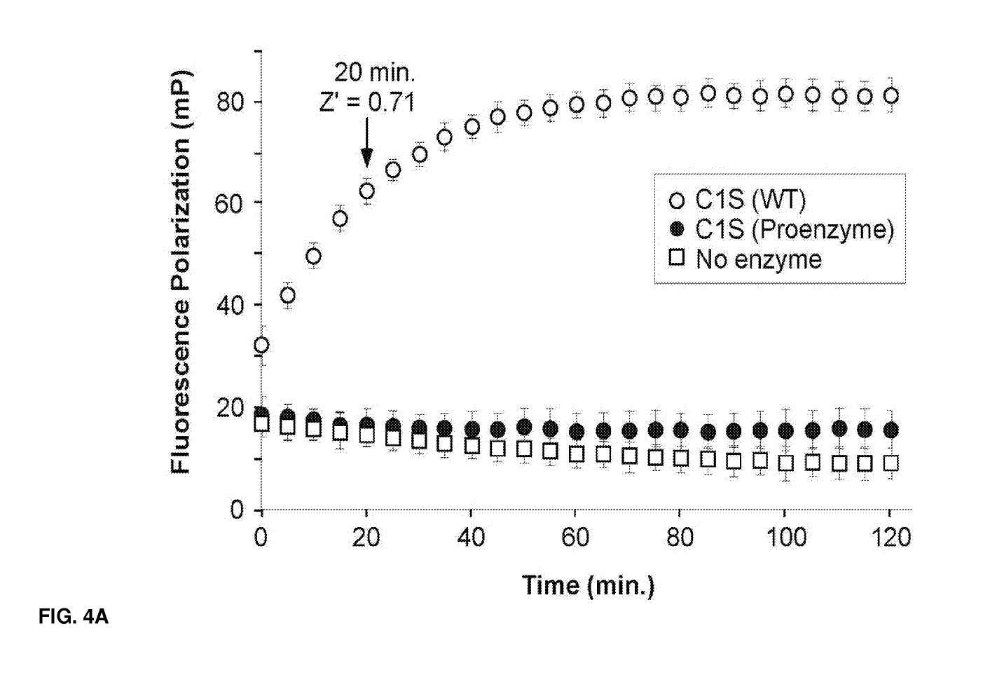

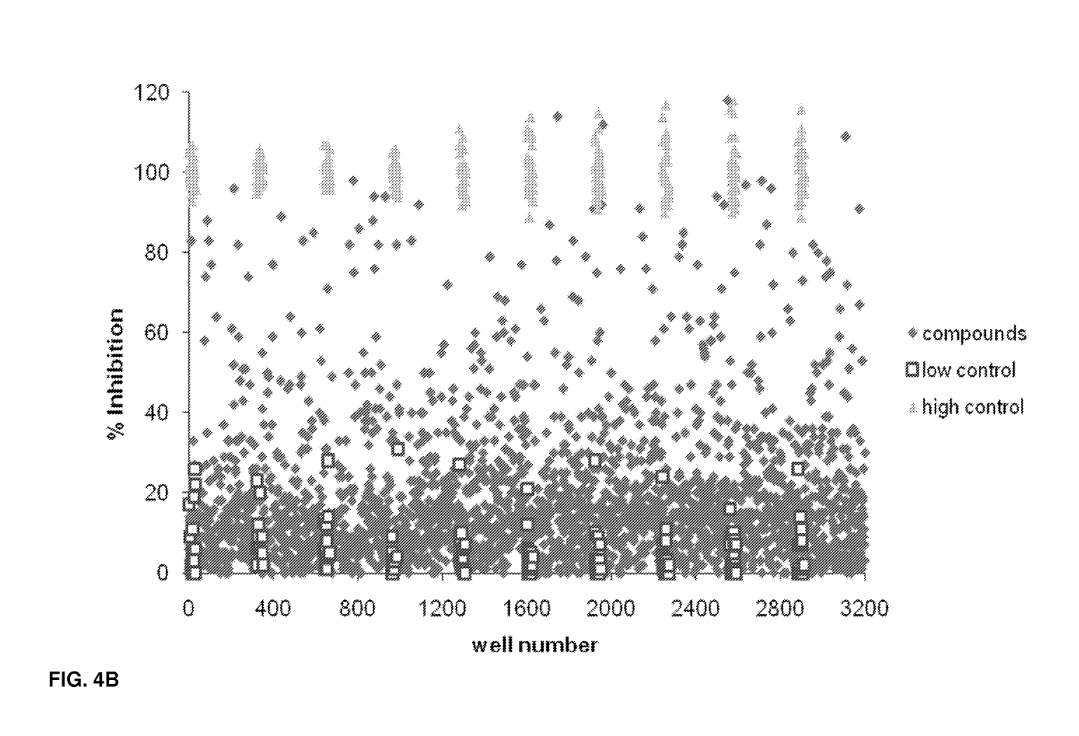

According to this disclosure, Z-factors may either be calculated as described above, i.e. by using experimental iterations involving a positive control compound, such as a known protease inhibitor, and randomized compounds of unknown activity. Alternatively, Z-factors may be calculated based on experimental iterations that do not involve any compound additions (See, e.g., FIGS. 3 and 4; Example 3). According to this disclosure, positive control experiments (indicating complete inhibition of the protease) may be designed, for example by applying an inactive protease to the assay, such as the zymogen form of a serine protease, or by omitting the protease entirely from the experiment. Similarly, negative control experiments (indicating full protease activity) may be designed by omitting the test compound of unknown activity, i.e., by contacting the protease with the molecular probe in the absence of a test compound. The HTS-assays of this disclosure are characterized by a Z-factor greater than 0.5, 0.6, 0.7, 0.8, or 0.9. In preferred embodiments, the Z-factor is greater than 0.7.

Certain aspects of the present disclosure further relate to complement serine protease inhibitors identified by the present disclosure. Complement serine protease inhibitors identified by the methods of the present disclosure may include, without limitation, serine protease inhibitors that contain a carbamate chemotype, an acyl-pyrazole chemotype, or a thiophenyl functionality. In some embodiments, complement serine protease inhibitors identified by the methods of the present disclosure may include, without limitation, hydroquinazoline derivatives and hydroquinazoline derivatives.

Examples of complement serine protease inhibitors identified by the methods of the present disclosure are described herein and include, without limitation, CD00825, GK00797, KM09391, PHG00507, and JFD00044.

### Methods of Treatment

The complement serine protease inhibitors identified through the methods of this invention can be used to treat disease conditions associated with or otherwise resulting from the overactivation of the complement system. Exemplary disease conditions include inflammatory disease conditions in humans and animals, including rheumatoid arthritis or ischaemia/reperfusion injury. Additional exemplary disease conditions include autoimmune diseases, neurodegenerative diseases and cancer. Exemplary disease conditions further include animal models of human diseases, such as the Arthus reaction or the reverse passive Arthus reaction. Exemplary inhibitors include small molecules comprising a carbamate chemotype, acyl-pyrazoles, or compounds containing a thiophenoyl functionality. Additional exemplary inhibitors include hydroquinazoline and hydroquinazoline derivatives. Additional exemplary inhibitors include compounds from the Maybridge compound collection, including CD00825, GK 00797, KM09391, PHG00507, and JFD00044.

Alternatively, serine protease inhibitors identified through the methods of this invention that are directed at inhibitory complement serine proteases, such as FI, can be used to activate the complement system. Such compounds can be used to treat disease conditions associated with or otherwise resulting from complement deficiencies or to treat disease conditions where complement activation may form a part of the therapeutic strategy. The complement deficiencies may have, e.g., a genetic or an environmental basis, or result secondarily from another disease condition. Complement deficiencies may include, without limitation, autoimmune disorders such as systemic lupus erythematosus and immune complexes disorders such as, glomerulonephritis including membranoproliferative glomerulonephritis type II. Disease conditions where complement activation may form a part of the therapeutic strategy may include, without limitation, certain bacterial infections, such as infections with encapsulated bacteria, including Neisseria meningitides, Streptococcus pneumoniae, Haemophilus influenzae, and Neisseria gonorrhoeae.

As used herein “therapeutically effective amount or dose,” of an agent, e.g., a pharmaceutical formulation, refers to an amount effective, at dosages and for periods of time necessary, to achieve the desired therapeutic or prophylactic result. An effective dosage can be administered in one or more administrations. For purposes of this invention, an effective dosage of drug, compound, or pharmaceutical composition is an amount sufficient to accomplish prophylactic or therapeutic treatment either directly or indirectly. As is understood in the clinical context, an effective dosage of a drug, compound, or pharmaceutical composition may or may not be achieved in conjunction with another drug, compound, or pharmaceutical composition. Thus, an “effective amount or dosage” may be considered in the context of administering one or more therapeutic agents, and a single agent may be considered to be given in an effective amount if, in conjunction with one or more other agents, a desirable result may be or is achieved.

In some embodiments, “treatment” may refer to clinical intervention designed to alter the natural course of the individual being treated during the course of clinical pathology. Desirable effects of treatment may include, without limitation, decreasing the rate of progression, ameliorating or palliating the pathological state, and remission or improved prognosis of a particular disease, disorder, or condition. In some embodiments, an individual may be successfully “treated”, for example, if one or more symptoms associated with a particular disease, disorder, or condition are mitigated or eliminated.

In some embodiments, “preventing” may include providing prophylaxis with respect to occurrence or recurrence of a particular disease, disorder, or condition in an individual. An individual may be predisposed to, susceptible to a particular disease, disorder, or condition, or at risk of developing such a disease, disorder, or condition, but has not yet been diagnosed with the disease, disorder, or condition. In some embodiments, an individual “at risk” of developing a particular disease, disorder, or condition may or may not have detectable disease or symptoms of disease, and may or may not have displayed detectable disease or symptoms of disease prior to the treatment methods described herein. In some embodiments, “at risk” may refer to an individual that has one or more risk factors, which are measurable parameters that correlate with development of a particular disease, disorder, or condition, as known in the art. In some embodiments, an individual having one or more of these risk factors has a higher probability of developing a particular disease, disorder, or condition than an individual without one or more of these risk factors.

In some embodiments, a “subject” for purposes of treatment, prevention, or reduction of risk may refer to any animal classified as a mammal, including humans, domestic and farm animals, and zoo, sport, or pet animals, such as dogs, horses, rabbits, cattle, pigs, hamsters, gerbils, mice, ferrets, rats, cats, and the like. In certain embodiments, the subject is human.

In some embodiments, the term “about” may refer to the usual error range for the respective value readily known to the skilled person in this technical field. Reference to “about” a value or parameter herein includes (and describes) embodiments that are directed to that value or parameter per se.

In some embodiments, the singular forms “a,” “an,” and “the” include plural reference unless the context clearly indicates otherwise. For example, reference to an “inhibitor of a complement serine protease” is a reference to from one to many inhibitors, such as molar amounts, and includes equivalents thereof known to those skilled in the art, and so forth.

It is understood that aspect and embodiments of the invention described herein include “comprising,” “consisting,” and “consisting essentially of” aspects and embodiments.

The invention will be more fully understood by reference to the following Examples. They should not, however, be construed as limiting the scope of the invention. All citations throughout the disclosure are hereby expressly incorporated by reference.

### EXAMPLES

This example illustrates the development of a robust high-throughput screening assay for complement serine proteases. Specifically, the development of a fluorescence-polarization-activity-based protein profiling (fluopol-ABPP) assay for the human complement serine protease C1S is illustrated. C1S is an 80 kDa serine protease that mediates the proteolytic activity of the C1 complement complex by cleaving both C2 and C4.

In general, fluopol-ABPP assays involve the reaction of a serine hydrolase with a probe containing a reactive group, such as a fluorophosphonate group, that specifically and covalently labels the active-site serine of enzymatically active serine hydrolases. These probes can further include additional tags for fluorescence detection, such as the rhodamine (Rh or TMRA) fluorophore. Specifically fluorescence polarization is a possible readout for quantifying small molecule interactions with macromolecular targets, such as serine proteases.

When excited with plane-polarized light, a fluorophore emits light parallel to the plane of excitation unless it rotates in the excited state. The speed of molecular rotation and resulting extent of depolarization are inversely proportional to molecular volume. Typically, small fluorophores (<10 kDa) rotate quickly and emit depolarized light (low FP signal) when free in solution, but rotate more slowly and emit highly polarized light (high FP signal) when bound to a large molecule (e.g., a protein). The reaction between a small-molecule activity-based probe and an enzyme results in a time-dependent increase in FP signal, thereby enabling the real-time monitoring of enzymatic activity in a homogeneous assay format.

The fluopol-ABPP assay development process for C1S proceeded in two steps. First, the protease activity and specificity of C1S labeling with a fluorophosphonate probe was confirmed in a low-throughput gel-based assay. In the second step, the C1S fluopol-ABPP assay was adapted to a homogeneous microtiter-plate-based HTS format and a first validation screen was conducted.

### Example 1

### A Low-Throughput Gel-Based Fluorescence Intensity Readout Confirms the Enzyme Activity Dependent Labeling of C1S and C1R with Fluorophosphonate-Rhodamine (FP-Rh)

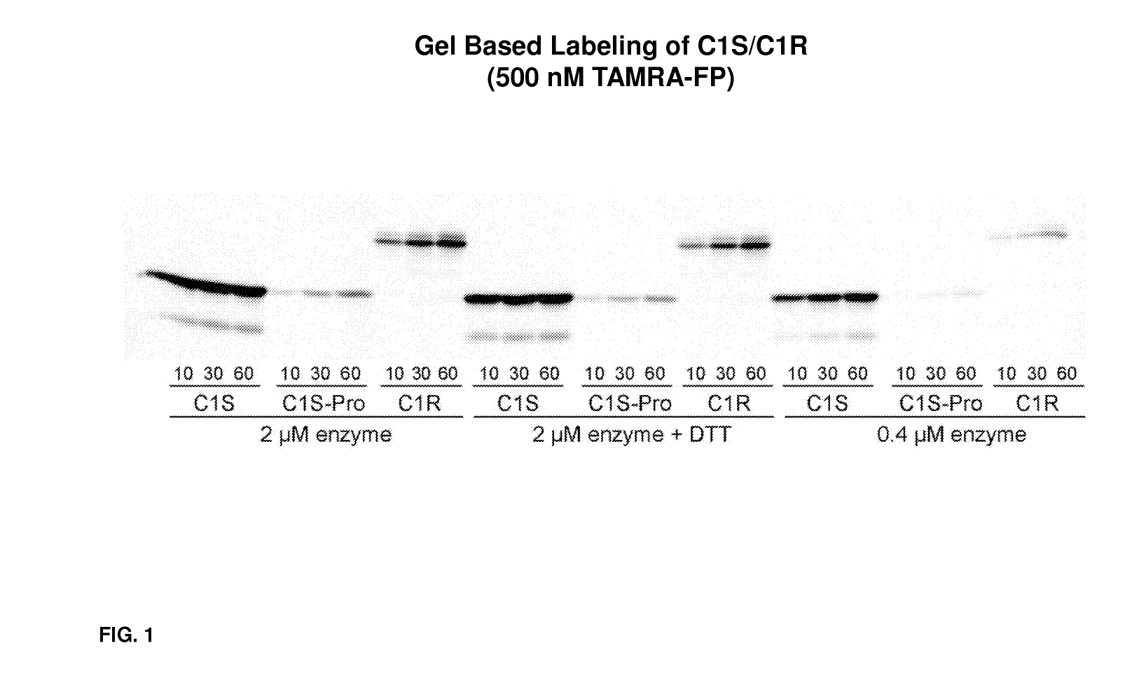

FIG. 1 shows the results of a gel-based fluorophosphonate-rhodamine (FP-Rh, also referred to as TAMRA-FP) labeling experiment. The complement serine proteases C1S and C1R and the catalytically inactive proenzyme C1S-Pro were incubated at concentrations of either 0.4 μM or 2 μM with 500 nM FP-Rh. Samples were taken after 10 min, 30 min, and 60 min and run on a 10% PAGE gel. The reaction buffer was 50 mM Sodium Phosphate pH 7.2, 130 mM NaCl, 0.01% Pluronic F-127. The gels were scanned as described in Patricelli et al., Proteomics, 2001, 1, 1067-1071. Briefly, labeled samples were visualized on a Hitachi FMBio IIe flatbed fluorescence scanner (MiraiBio, Alameda, Calif., USA) with excitation provided by the 532 nm line of a 50 mW neodymium-doped yttrium-aluminum-garnet (Nd.:YAG) laser. A 605 nm bandpass filter was used to detect FP-TMR.

The experiment illustrates that serine protease labeling with FP-Rh occurred in a time- and enzyme activity dependent manner. The active protease C1S at 2 μM showed strong FP-Rh labeling already after 10 min, whereas labeling of the inactive proform C1S-Pro was essentially absent even after an extended 60 min reaction time. It is noted that active C1S (a dimer) and its proenzyme C1S-Pro have the same molecular weight. C1S activation results from cleavage of the C1s proenzyme; no amino acids are lost in the process, but the resulting C1S chains remain linked. Time-dependent labeling of C1S was clearly detectable also at the lower enzyme concentrations of 0.4 μM. By comparison, under the chosen reaction conditions, C1R labeling was found to be much weaker than C1S labeling at 2 μM and was barely detectable at 0.4 μM. Both C1S and C1R labeling was found to be unaffected by 1 mM DTT, demonstrating that the FP-Rh label did not non-specifically modify surface exposed cysteine residues in C1S and C1R.

In conclusion, the gel-based labeling experiment demonstrates the specificity and enzyme activity dependence of C1S and C1R labeling with the fluopol-ABPP probe FP-Rh. Moreover, because in SDS-PAGE gels small molecule test compounds are readily separated from their macromolecular targets, the gel-based C1S and C1R activity assay is useful as a secondary assay to rule out a subset of false-positive or nonselective primary hits that are routinely found in large-scale high-throughput screens.

### Example 2

### FP-Rh Labeling of C1S can be Followed by Fluorescence Polarization in a Homogenous, High-Throughput Compatible Format

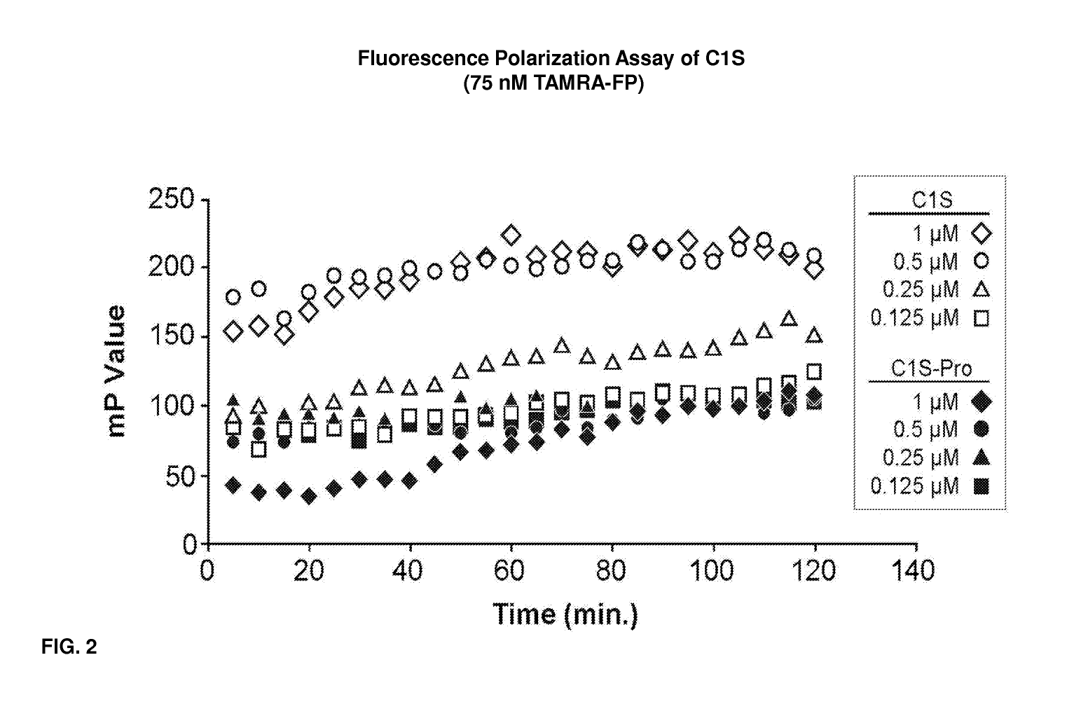

FIG. 2 shows an experiment demonstrating that FP-Rh labeling of C1S can be followed by fluorescence polarization readouts in a homogeneous, high throughput-compatible assay format. The experiment was conducted in a 384-well plate. In this experiment, C1S concentration and reaction times were varied to determine a range of possible fluopol-ABPP assay conditions. The results show that time- and C1S activity dependent, fluorescence polarization signals were obtained at C1S concentrations of 0.5 μM and 1.0 μM.

### Example 3

### Application of the C1S Fluopol-ABPP Assay in a Pilot High-Throughput Screen

FIG. 3 shows an exemplary microtiter plate layout for a fluopol-ABPP HTS assay for C1S.

Briefly, 10 μl of a 0.55 μM C1S solution (0.5 μM final) in assay buffer (50 mM sodium phosphate, pH 7.2, 130 mM NaCal, 0.01% Pluronic F-127, 1 mM DTT) were dispensed into the negative control and test compound wells of columns 1-22 in a Greiner Bio-One 384-well plate (cat #784076). The remaining positive control wells in columns 23-24 received 10 μl assay buffer. Next, 50 nl test compound DMSO stocks (5 mM) were transferred to the test compound wells in columns 3-22; similarly 50 nl DMSO was transferred into the control wells in columns 1, 2, 23, and 24. Compounds and DMSO controls were then incubated with C1S or reaction buffer controls for 30 minutes. In the meantime, a 750 nM FP-TAM (tetramethylrhodamine) probe solution was prepared (immediately prior to use) by diluting a 50 μM DMSO stock solution (aliquotted and stored at −80° C.) 1:66.6 in assay buffer. 1.1 μl of the 750 nM FP-TAM solution were then dispensed into all wells of the 384-well plate, resulting in a final probe concentration of 75 nM. Depending on the experiment, the microtiter plate was then read either continuously or, alternatively, read in an endpoint format following 20 min incubation. Plate readings were taken on a Perkin Elmer Envision reader, using the Optimized Bodipy TMR FP Dual Emission Label 2100-8070 filter settings (consisting of the following filters and mirror modules: Bodipy TMR FP Dual Minor Module (2100-4080), Bodipy TMR FP Excitation Filter (2100-5050), Bodipy TMR FP Emission Filter S-pol (2100-5160), and Bodipy TMR FP Emission Filter P-pol (2100-5170).

FIG. 4 shows results of C1S fluopol-ABPP assays conducted in a 384-well plate.

FIG. 4A shows a time-course taken in the absence of compounds. The fluorescence polarization signal was found to increase in a time-dependent and C1S activity-dependent manner. Moreover, this experiment shows that a robust C1S assay performance can be achieved in a 384-well plate, as indicated by tight error bars (indicating standard deviations across representative wells) and robust Z′ values of 0.71 after 20 min incubation.

FIG. 4B shows the results of a pilot screen of the NIH Validation Set compound collection (2 mM DMSO stocks, 5-10 μM final concentration) using the 384-well plate C1S fluopol-ABPP assay. In this graph, the inhibitory activity of each test compound is shown as percent inhibition of C1S enzyme activity. In this pilot screen negative control wells (low control) contain C1S enzyme and DMSO, but no test compound. Thus, positive control wells reflect assay results that are expected in the presence of a completely inactive test compound. By contrast, positive control wells (high control) are designed to indicate assay results expected in the presence of strong inhibitors that entirely abolish C1S activity. Accordingly, to mimic the state of complete C1S inhibition, the enzyme was omitted from the positive control wells.

The pilot screen results demonstrate that small molecule hits can be readily identified in the 384-well C1S fluopol-ABPP assay. Some of these compounds were found to score activities in the assay that are consistent with the complete inhibition of C1S.

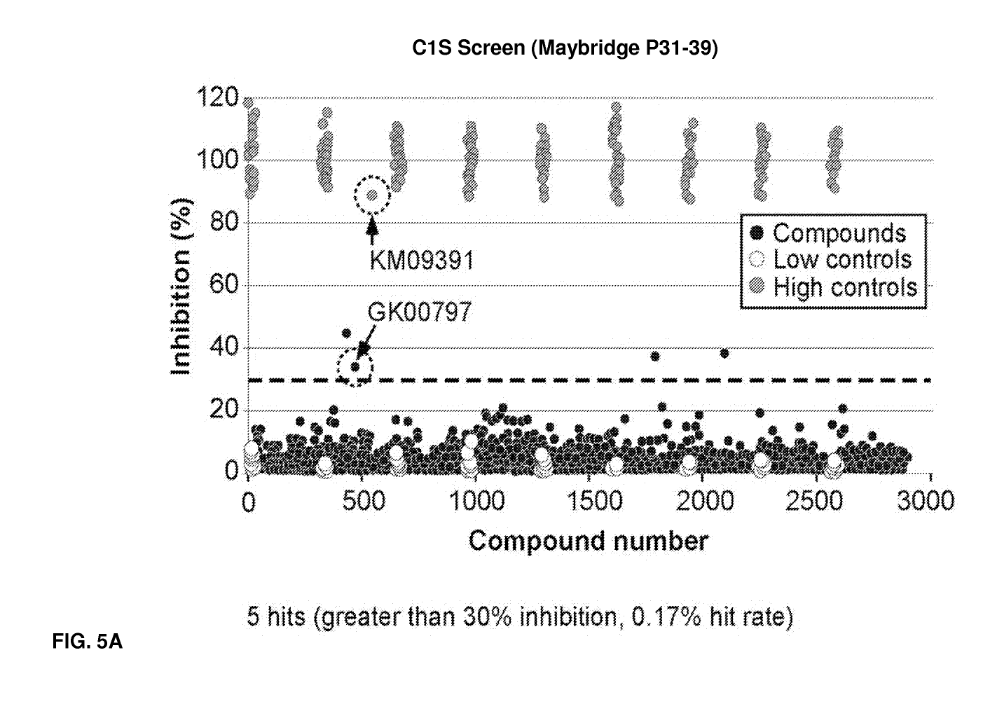

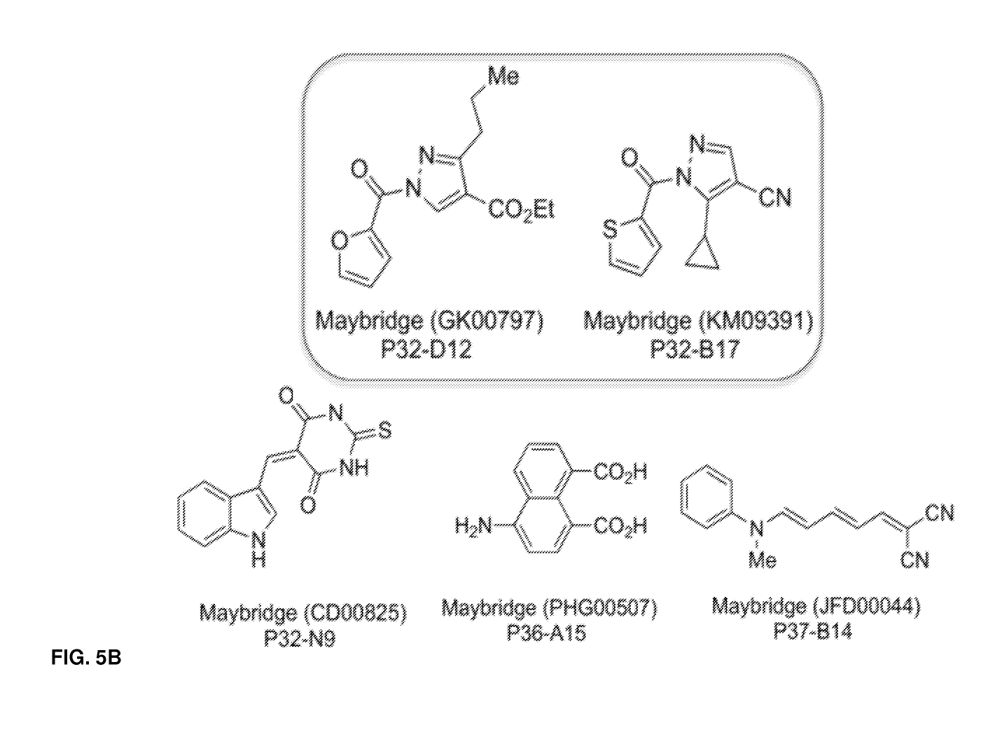

An additional 384-well C1S fluopol-ABPP screen was run against compounds from the Maybridge compound collection (FIG. 5A). Five hits were identified that inhibited C1S activity by more than 30% (yielding a 0.17% hitrate). The structures of these compounds are shown in FIG. 5B.

### Example 4

### Secondary Hit Validation in Low-Throughput Gel-Based Assay

Following primary screens of the NIH Validation Set and the Maybridge compounds, two of the identified hits from the Maybridge collection (GK00797 and KM09391) were followed up in secondary assays for hit validation. As one of the secondary assays, the low-throughput gel-based assay of Example 1 was used.

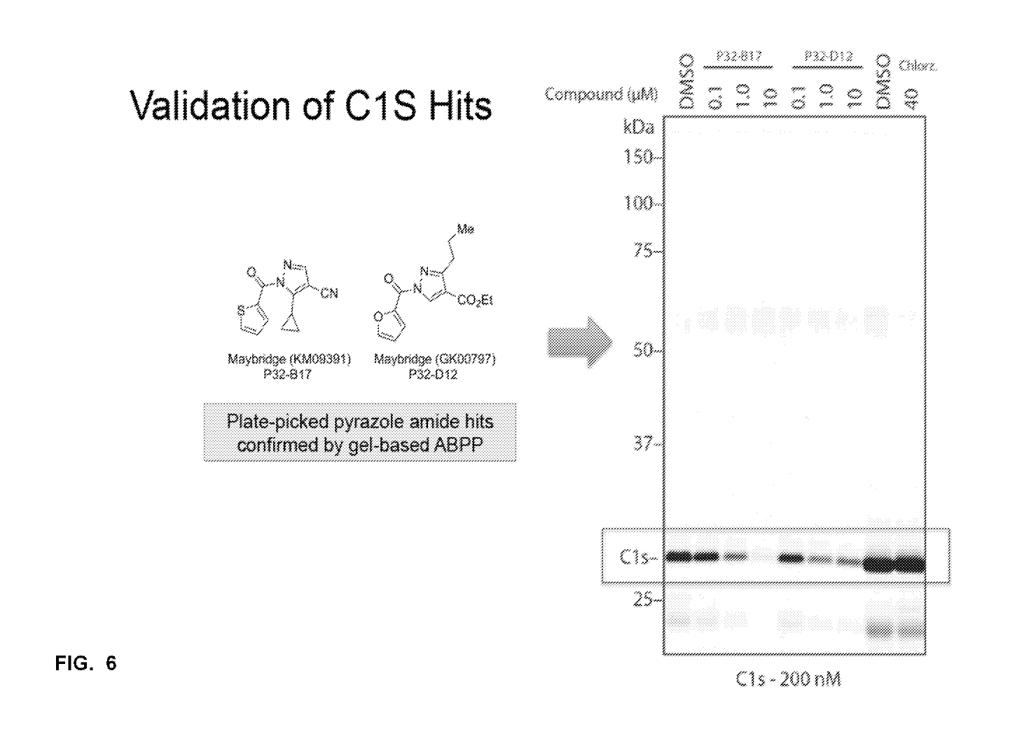

Both compounds, GK00797 and KM09391, inhibited C1S in the secondary gel-based assay and were therefore considered validated HTS hits (FIG. 6).

### REFERENCES

1. Sim, R. B. & Laich A., Serine proteases of the complement system. Biochem. Soc. Trans. 28, 545-550 (2000).
2. Sim, R. B. & Tsiftsoglou, S. A., Proteases of the complement system. Biochem. Soc. Trans. 32, 21-27 (2004).
3. Gál P., Dobó J., Beinrohr L., Pál G. & Závodszky P., Inhibition of the Serine Proteases of the Complement System. Adv. Exp. Med. Biol. 734, 23-40 (2013).
4. Bachovchin D. A., Brown S. J., Rosen H. & Cravatt B. F., Identification of selective inhibitors of uncharacterized enzymes by high-throughput screening with fluorescent activity-based probes. Nat. Biotechnol. 27, 387-394 (2009).
5. Bachovin D. A. et al., Discovery and optimization of sulfonyl acrylonitriles as selective covalent inhibitors of protein phosphatase methylesterase-1. J. Med. Chem. 54, 5229-5236 (2011).
6. Buerke M. et al., Novel small molecule inhibitor of C1s exerts cardioprotective effects in ischemia-reperfusion injury in rabbits. J. Immunol. 167, 5375-5380 (2001).
7. Szalai A. J. et al., The Arthus reaction in rodents: species-specific requirement of complement. J. Immunol. 164, 463-468 (2000).
8. Qu H., Ricklin, D., & Lambertis J. D., Recent developments in low molecular weight complement inhibitors. Mol. Immunol. 47, 185-195 (2009).
9. Catterall C. F., Lyons A., Sim R. B., Day A. J. & Harris T. J., Characterization of primary amino acid sequence of human complement control protein factor I from an analysis of cDNA clones. Biochem. J. 242, 849-856 (1987).
10. Malm S., Jusko M., Eick S., Potempa J., Riesbeck K., et al., Acquisition of Complement Inhibitor Serine Protease Factor I and Its Cofactors C4b-Binding Protein and Factor H by Prevotella intermedia. PLoS ONE 7(4): e34852 (2012).
11. Figueroa J. E. & Densen P., Infectious diseases associated with complement deficiencies. Clin Microbiol Rev. 4, 359-395 (1991).
12. Blom A. M., Complement: Deficiency Diseases. Published online: http://onlinelibrary.wiley.com/doi/10.1002/9780470015902.a0002154.pub2/full (2010).
13. Perlmutter D. H., Colten H. R., Grossberger D., Strominger J., Seidman J. G., Chaplin D. D. J., Expression of complement proteins C2 and factor B in transfected L cells. Clin. Invest. 76, 1449-54 (1985).
14. Schmidt C. Q., Slingsby F. C., Richards A., Barlow P. N., Production of biologically active complement factor H in therapeutically useful quantities. Protein Expr. Purif. 76, 254-63 (2011).
15. Verhelst S. H. L. & Bogyo M., Chemical Proteomics Applied in Target Identification and Drug Discovery, BioTechniques 38, 175-177 (2005).
16. Lin C. 6HCV NS3-4A Serine Protease, Chapter 6 in Hepatitis C Viruses: Genomes and Molecular Biology. Tan S. L., editor. Norfolk (UK), Horizon Bioscience (2006)
17. Baker S. Z., Ding C. Z., et al., Therapeutic Potential of Boron Containing Compounds, Future Med. Chem. 1, 1275-1288 (2009)
18. Li X., Zhang Y. K. et al., Design, Synthesis and SAR of α-Amino Cyclic Boronate-containing Macrocyclic Inhibitors of HCV NS3/4A Serine Protease, Poster No. MEDI 126 of 239th ACS National Meeting, San Francisco, Mar. 21, 2010.

1. A method of screening for inhibitors of a complement serine protease, the method comprising:
a) contacting a complement serine protease with a molecular probe in the presence and absence of a test compound; and
b) measuring the level of interaction of the serine protease with the molecular probe, wherein a reduction in the interaction in the presence of the test compound compared to the absence of the test compound indicates that the test compound is an inhibitor of the serine protease.

2. The method of claim 1, wherein the serine protease is contacted with at least 100, 500, 1,000, 5,000, 10,000, 50,000, 100,000, 500,000, 1,000,000, 1,500,000, 2,000,000, 2,500,000, or 3,000,000 test compounds within a 24 hour time period.

3. The method of claim 1, wherein the method comprises an assay Z-factor value that is greater than 0.5, 0.6, 0.7, 0.8, or 0.9.

4. The method of claim 1, wherein inhibition of the serine protease inhibits the complement cascade.

5. (canceled)

6. The method of claim 1, wherein the serine protease is selected from C1s, C1r, MASP-1, MASP-2, MASP-3, C2, C2a, C4bC2a, C4b2a3b, C3bBbC3b, C3 convertase, C5 convertase, Factor 2a, Factor Bb, Factor D, and Factor I.

7. (canceled)

8. (canceled)

9. (canceled)

10. The method of claim 1, wherein the serine protease is provided in an inactive form and activated prior to execution of step b).

11. (canceled)

12. (canceled)

13. The method of claim 1, wherein the molecular probe binds the active site of the serine protease in a non-covalent manner.

14. The method of claim 1, wherein the molecular probe binds the active site of the serine protease and forms a covalent bond with the serine protease.

15. (canceled)

16. The method of claim 15, wherein the fluorophore is selected from a fluorescent protein, a fluorophosphonate (FP) group, a non-protein organic fluorophore, a quantum dot, or a peptide.

17-22. (canceled)

23. The method of claim 1, wherein the molecular probe comprises a non-fluorescent detection moiety.

24-26. (canceled)

27. The method of claim 1, wherein the protease is contacted with the molecular probe in a homogeneous phase.

28. The method of claim 1, wherein the protease is contacted with the test compound first and the molecular probe second.

29. The method of claim 1, wherein the protease is contacted with the test compound and the molecular probe at the same time.

30. The method of claim 1, wherein the test compound is at least two test compounds.

40. (canceled)

41. The method of claim 1, wherein the test compound is a control compound.

42. (canceled)

43. The method of claim 1, wherein the serine protease is contacted with a molecular probe immobilized on a surface.

35-42. (canceled)

43. A complement serine protease inhibitor identified by the method of claim 1.

44. The complement serine protease inhibitor of claim 43, wherein the complement serine protease inhibitor comprises a carbamate chemotype, an acyl-pyrazole chemotype, or a thiophenyl functionality.

45. (canceled)

46. A method of treating a disease condition associated with the excessive activation of the complement system in a subject in need of such treatment, the method comprising the step of administering a therapeutically effective dose of a complement serine protease inhibitor of claim 43, wherein the complement serine protease inhibitor inhibits activation of the complement system.

47-50. (canceled)

51. A method of treating a disease condition associated with complement deficiency in a subject in need of such treatment, the method comprising the step of administering a therapeutically effective dose of a complement serine protease inhibitor of claim 43, wherein the complement serine protease inhibitor in an inhibitor of Factor I.

52-56. (canceled)
